(19) United States
(12) Plant Patent Application Publication
(10) Pub. No.: US 2004/0244084 P1
(43) Pub. Date: Dec. 2, 2004

(54) Campanula plant named '08.01.17'

(75) Inventors: Gert Kim Jensen, Odense N (DK); Per Elmegaard Andersen, Skalskor (DK)

Correspondence Address: FOLEY AND LARDNER, SUITE 500, 3000 K STREET NW, WASHINGTON, DC, 20007, US

(73) Assignees: Gartneriet PKM ApS; Gartneriet Elmegaard Andersen

(21) Appl. No.: 10/648,304

(22) Filed: Aug. 27, 2003

(30) Foreign Application Priority Data
May 26, 2003 (QZ) 2003/0982

(51) Int. Cl. A01H 5/00

(52) U.S. Cl. PLT/263

(57) ABSTRACT

A distinct cultivar of Campanula plant named ‘08.01.17’, characterized by having compact, upright plant habit; dense and bushy plant form mainly due to more upright stems; vigorous growth habit and less need for chemical growth retardation; greater number of flowers per plant; large upright blue flowers, and no need for vernalization.

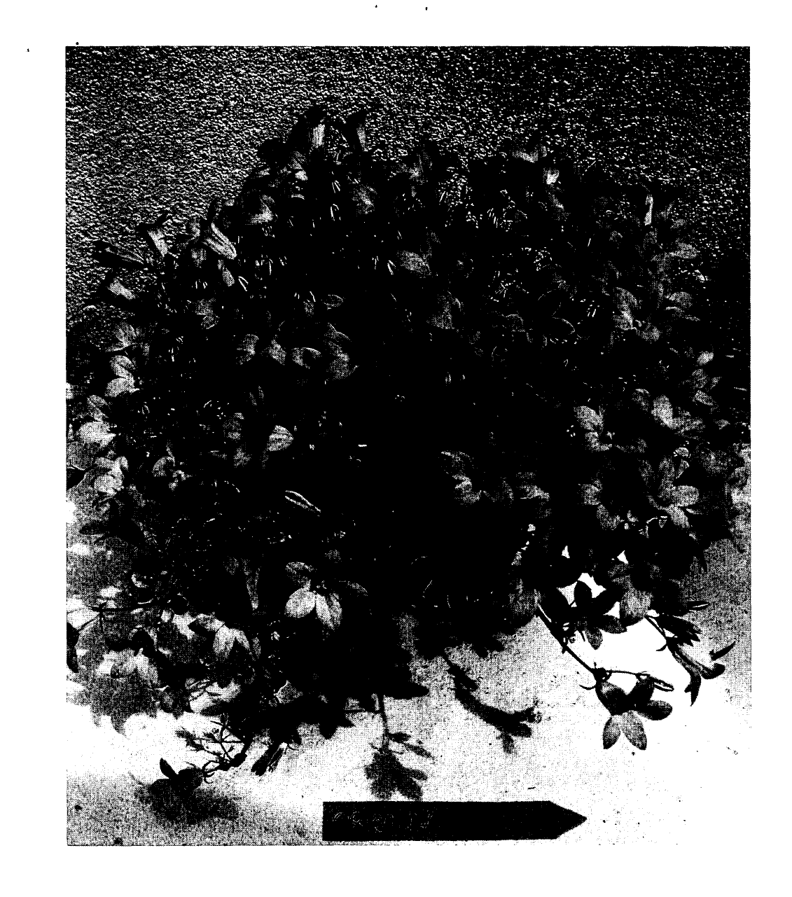

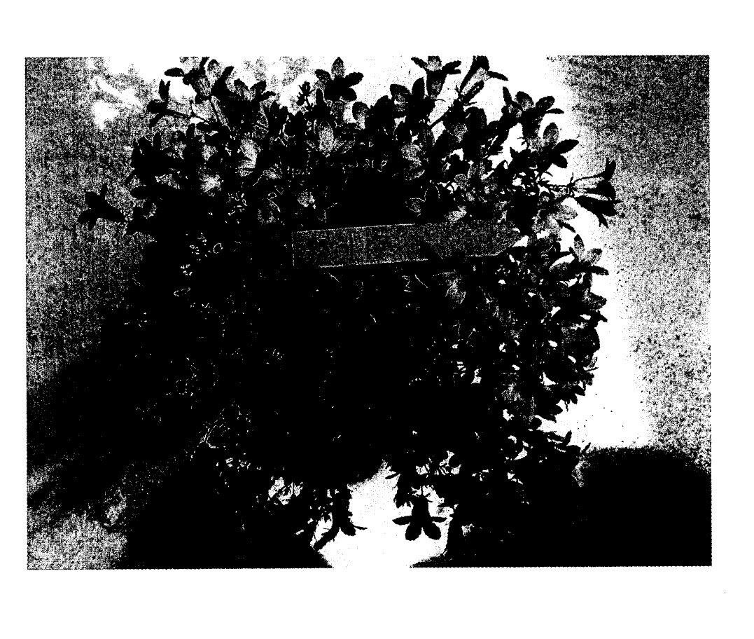

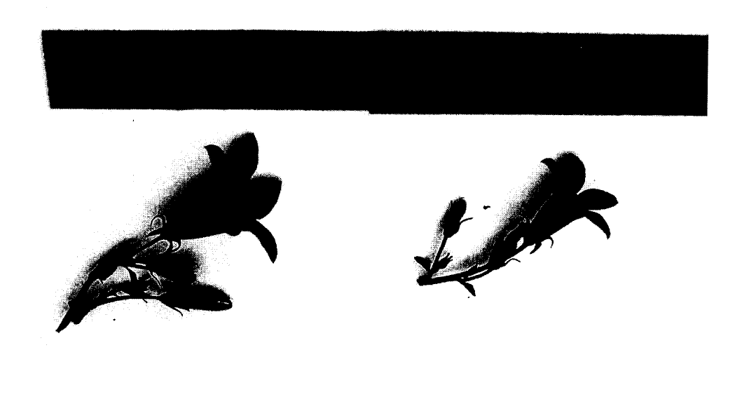

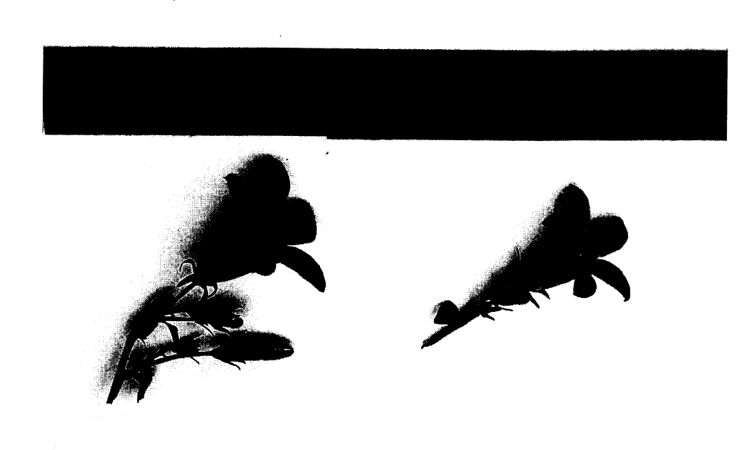

### LATIN NAME OF THE GENUS AND SPECIES OF THE PLANT CLAIMED

Campanula portenschlagiana

### VARIETY DENOMINATION

‘08.01.17’

### BACKGROUND OF THE INVENTION

The present Invention relates to a new and distinct cultivar of Campanula plant, botanically known as Campanula portenschlagiana Schult., commonly known as Dalmatian Bellflower, and hereinafter referred to by the name ‘08.01.17’.

The new Campanula is a product of a planned breeding program conducted by the Inventors, Gert K. Jensen and Per Elmegaard Andersen, in Shus, Denmark. The new Campanula originated from a cross made in 2001 by the Inventors between a proprietary selection of Campanula portenschlagiana Schult named ‘08.08’ (unpatented) as the female parent, and a proprietary selection of Campanula portenschlagiana Schult named ‘08.98.05’ (unpatented) as the male parent. The Inventors selected the new Campanula cultivar from the progeny of the above crossing in 2001 on the basis of its compact and freely flowering habit. Plants of the new Campanula are more upright, compact and more freely flowering than plants of both parental selections.

Asexual reproduction of the new cultivar by terminal cuttings taken and propagated in Shus, Denmark, has shown that the unique features of this new Campanula are stable and reproduce true to type in many successive generations.

### SUMMARY OF THE INVENTION

Plants of the cultivar ‘08.01.17’ have not been observed under all possible environmental conditions. The phenotype may vary somewhat with variations in environment such as temperature, light intensity, day length, and fertility level without, however, any variance in genotype.

The following traits have been repeatedly observed and are determined to be the unique characteristics of ‘08.01.17’. These characteristics in combination distinguish ‘08.01.17’ as a new and distinct cultivar:

1. Compact, upright plant habit;

2. Dense and bushy plant form, mainly due to more upright stems;

3. Vigorous growth habit, less need for chemical growth retardation;

4. Greater number of larger flowers per plant;

5. Large upright blue flowers; and

5. No need for vernalization.

Side-by-side comparisons between the instant plant and the parental cultivars, ‘08.08. and ‘08.988.05’, were conducted by the Inventors in Stige Denmark. Plants of ‘08.01.17’ differ from the cultivars ‘08.08’ and ‘08.988.05’ in the following characteristics:

1. Plants of ‘08.01.17’ have shorter internodes and shorter leaves than plants of the cultivars ‘08.08’ and ‘08.98.05’.

2. Plants of ‘08.01.17’ have light gray-green colored leaves whereas plants of the cultivars ‘08.08’ and ‘08.98.05’ have green-colored leaves.

3. Plants of ‘08.01.17’ have shorter flower peduncles than plants of the cultivars ‘08.08’ and ‘08.98.05’.

4. Plants of ‘08.01.17’ are shorter and more compact than plants of the cultivars ‘08.08’ and ‘08.98.05’.

5. Plants of ‘08.01.17’ have more flowers per plant and larger flowers than plants of the cultivars ‘08.08’ and ‘08.98.05’.

### BRIEF DESCRIPTION OF THE PHOTOGRAPHS

The accompanying photographic drawings illustrate the overall appearance of the new cultivar, showing the colors as true as it is reasonably possible to obtain in colored reproductions of this type. Colors in the photographs may differ slightly from the color values cited in the detailed botanical description, which more accurately describe the actual colors of ‘08.01.17’.

The first photographic drawing shows a side perspective view of a typical flowering plant of ‘08.01.17’ compared to the parental cultivars ‘08.08’ and ‘08.98.05’, as grown in 11 cm pots. The second photographic drawing shows a close-up view of typical flowering racemes of ‘08.01.17’ along ‘08.08’ and ‘08.98.05’.

### DETAILED BOTANICAL DESCRIPTION

In the following description, color references are made to The Royal Horticultural Society (RHS) Colour Chart, 4^{th} edition. Plants were grown under greenhouse conditions. The plants described were about 14 weeks old after cutting, as grown in 11 cm pots.

Botanical classification: Campanula portenschlagiana Schult.

Parentage:

Female parent.—Campanula portenschlagiana Schult. ‘08.08’.

Male parent.—Campanula portenschlagiana Schult. ‘08.98.05’.

Propagation: Terminal vegetative cuttings.

Time to initiate roots.—About 10 to 14 days at 18 to 21 C. in tunnels in a greenhouse.

Root description.—Fine, well branched.

Plant description:

Form.—Perennial, herbaceous plant with upright plant habit, and overall globular shape with upright stems. Campanulate flowers in racemes. Freely branching with lateral branches forming at every node; dense and bushy.

Crop time.—After rooting, about 14 weeks are required to produce finished flowering plants in 11 cm pots.

Plant height (soil level to top of plant plane).—About 13 cm.

Plant spread.—23-27 cm.

Lateral branches.—Approximately 65-75, average length 11-13 cm, average branch diameter 1-2 mm, 5-7 leaves per lateral branch.

Internode length.—15 mm.

Stem.—RHS 143B in color.

Vigor.—Vigorous growth rate.

Foliage description: Leaves single, dentate, cordate, palmate venation. Length: 15-20 mm. Width: About 25 mm. Shape: cordate. Apex: broad acuminate. Base: cordate. Margin: broadly dentate. Texture: smooth, glabrous, dull. Short stiff hairs on abaxial side and along veins and margin. Color: Young foliage, upper and lower surfaces: 137A, green. Mature foliage, upper surface: N 139C, lower surface 139B. Venation, 138D, palmate venation pattern.

Petiole.—4-6 cm in length, 1-2 mm diameter, RHS N138D in color.

Flower description:

Flower arrangement and shape.—Single, upright flowers in racemes; campanulate flowers with small star shaped calyx.

Natural flowering season.—Continuous throughout the spring and summer. Season can be extended by vernalization and long day treatments.

Flower longevity on the plant.—Longevity of individual flowers is highly dependent on temperature and light conditions. Flowers persistent.

Number of flowers per inflorescence.—4-5.

Inflorescence size.—Length: about 11 cm.

Flowers.—Length: about 20 mm. Diameter: about 25 mm. Lanceolate, acuminate petal lobes: 10 mm long and 7 mm wide. Corolla color: blue, upper surfaces N87B, lower surface N87C, white lines along fusion margins of petals.

Buds.—Length: up to 15 mm, diameter: up to 4 mm, oblong, ridged shape, color: white to N88B.

Petals.—Arrangement: single, sympetalous, campanulate; 5 in number, basally fused, entire margin, acute apex, length 8-10 mm, width: 7 mm, color when opening RHS N87D, color when fully opened: RHS N87C; velvety texture.

Sepals.—Shiny, glabrous, free arrangement, 5 in number, cuspidate apex, fused base, length: 3 mm, width: 1 mm, color: immature RHS 144B (both surfaces); mature RHS 138A (both surfaces).

Peduncle.—Strength: moderately strong. Length: about 25 mm. Diameter: about 1 mm. Color: 138D light green.

Reproductive organs:

Stamen.—5, fused until pollen is shed.

Anther.—1 mm in size, color RHS 158B.

Pollen.—RHS 158B, average production.

Pistil.—1, 12 mm in length, tripartite shape.

Stigma.—Conspicuous tripartite, color: 85A blue.

Style.—Length: 11 mm, color RHS 84B.

Ovary.—RHS 150D.

Seed.—Length: about 1 mm. Diameter: about 0.3 mm.

Weather tolerance.—Plants of the new Campanula have exhibited good tolerance to draught, rain and wind, low temperature resistant to −15C.

I/We claim:

1. A new and distinct cultivar of Campanula plant named ‘08.01.17’, as illustrated and described herein.
